(19) United States
(12) Patent Application Publication
(10) Pub. No.: US 2026/0306078 A1
(43) Pub. Date: Oct. 8, 2026

(54) Collapsible Funnel with Integrated Mesh Screen for Capturing Kidney Stones from Urine Flow

(71) Applicant: Gavin S Ward, Indianapolis, IN (US)

(72) Inventor: Gavin S Ward, Indianapolis, IN (US)

(21) Appl. No.: 19/642,587

(22) Filed: Apr. 8, 2026

Related U.S. Application Data
(60) Provisional application No. 63/785,164, filed on Apr. 8, 2025.

(51) Int. Cl. A61B 10/00; B01D 29/05; G01N 1/10; G01N 1/20

(52) U.S. Cl. CPC A61B 10/007; B01D 29/05; G01N 1/20; B01D 2201/0415; B01D 2201/184; G01N 2001/1006

(57) ABSTRACT

A fluid straining apparatus configured to capture particulate matter from a fluid is disclosed. The apparatus includes an outermost ring portion defining a top frame, a plurality of ring portions telescopically coupled to one another by sleeve portions, and an innermost ring portion coupled to a mesh filter. The ring portions are movable between a collapsed configuration for compact storage and an extended configuration for use. The mesh filter is configured to permit passage of fluid while retaining particulate matter. In some embodiments, the ring portions and sleeve portions are formed from flexible polymeric materials, including silicone or thermoplastic elastomers, to facilitate telescopic movement and structural retention. The mesh filter may be secured within a groove of the innermost ring portion or removably coupled thereto and may have a mesh size selected based on a desired filtration application.

### CROSS-REFERENCE TO RELATED APPLICATIONS

This application claims the benefit of U.S. Provisional Patent Application No. 63/785,164, filed on Apr. 8, 2025, the entirety of which is hereby incorporated by reference.

### FIELD OF THE DISCLOSURE

The present disclosure relates generally to medical collection and filtration devices, and more particularly to portable urine straining apparatuses configured to capture particulate matter from urine, such as kidney stones or other debris, for diagnostic or monitoring purposes.

### BACKGROUND

The collection and analysis of particulate matter from urine, such as kidney stones, crystals, or other debris, is commonly required for diagnostic and monitoring purposes in medical practice. Patients experiencing conditions such as nephrolithiasis are often instructed to strain urine to capture expelled stones or fragments for subsequent analysis. Such analysis can assist healthcare providers in determining stone composition and guiding treatment or preventative strategies.

Conventional urine straining devices are typically simple mesh filters, cups, or disposable strainers that are manually held or positioned during use. These devices often suffer from several limitations. For example, many existing strainers are bulky or rigid, making them inconvenient to carry or store, particularly for patients who require continuous monitoring outside of a clinical setting. Additionally, conventional devices may lack structural support or ergonomic features, making them difficult to position accurately relative to a urine stream.

Further, existing urine strainers may not provide sufficient filtration efficiency or durability, particularly when reused over extended periods. Disposable options, while convenient, can generate unnecessary waste and may not provide consistent performance. Reusable devices, on the other hand, may be difficult to clean or maintain and may not collapse into a compact form for storage.

Accordingly, there remains a need for a urine straining apparatus that is portable, compact, and easy to use, while providing reliable filtration of particulate matter. There is also a need for a device that can transition between a compact storage configuration and an expanded operational configuration, thereby improving convenience for users in both clinical and non-clinical environments.

### SUMMARY OF THE DISCLOSURE

The present disclosure provides a fluid straining apparatus configured to capture particulate matter from urine while allowing fluid to pass therethrough. The apparatus includes a plurality of telescopically coupled ring portions that are movable between a collapsed configuration and an extended configuration. In the collapsed configuration, the apparatus occupies a compact form factor suitable for storage and transport, while in the extended configuration, the apparatus provides an enlarged filtration area for effective urine straining.

An outermost ring portion defines a top frame configured to be positioned relative to a urine stream, while an innermost ring portion supports a mesh filter configured to retain particulate matter while permitting urine to pass through. The ring portions may be interconnected by sleeve portions that facilitate controlled telescopic movement between configurations. In some embodiments, the ring portions are formed from flexible or elastomeric materials, such as silicone, to improve durability and user comfort.

The apparatus may further include a tab 320 extending from the outermost ring portion, the tab 320 optionally defining an aperture configured to facilitate attachment to a support structure, such as a hook or key ring, thereby enhancing portability and usability.

The disclosure also provides methods of using the apparatus, including extending the ring portions from a collapsed configuration, positioning the apparatus relative to a urine stream, and directing urine through the mesh filter such that particulate matter is retained. After use, the apparatus may be collapsed for storage.

The disclosed apparatus provides a compact, reusable, and user-friendly solution for capturing particulate matter from urine, enabling improved monitoring and collection in clinical, home, or ambulatory settings.

### BRIEF DESCRIPTION OF DRAWINGS

FIG. 1 illustrates a top view of a fluid straining apparatus in a deployed configuration.

FIG. 2 illustrates a side view of the fluid straining apparatus in a deployed configuration.

FIG. 3 illustrates a side view of the fluid straining apparatus in a collapsed configuration.

FIG. 4 illustrates a perspective view of the fluid straining apparatus in a collapsed configuration.

FIG. 5 illustrates perspective views of the fluid straining apparatus.

### DETAILED DESCRIPTION

The following detailed description is provided to enable a person of ordinary skill in the art to make and use the disclosed subject matter. The description sets forth illustrative embodiments and is not intended to be limiting. It will be understood that various modifications, combinations, and alternatives may be made without departing from the spirit of the disclosure. Accordingly, the embodiments described herein are to be considered illustrative in nature.

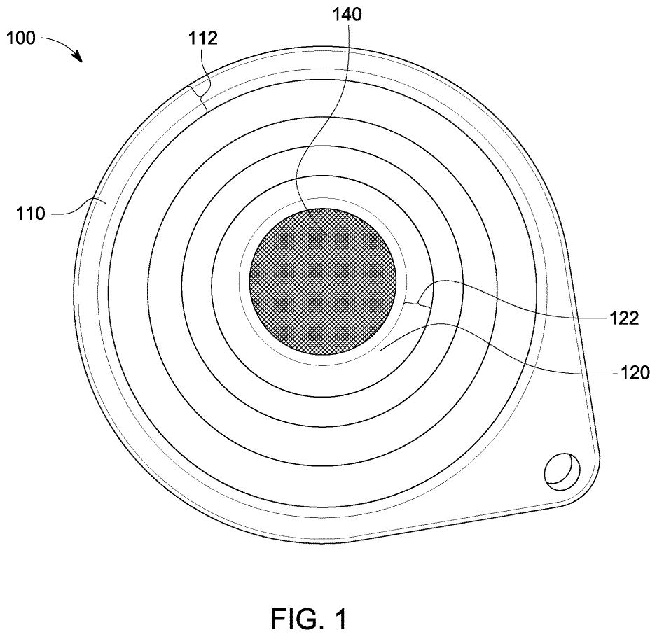

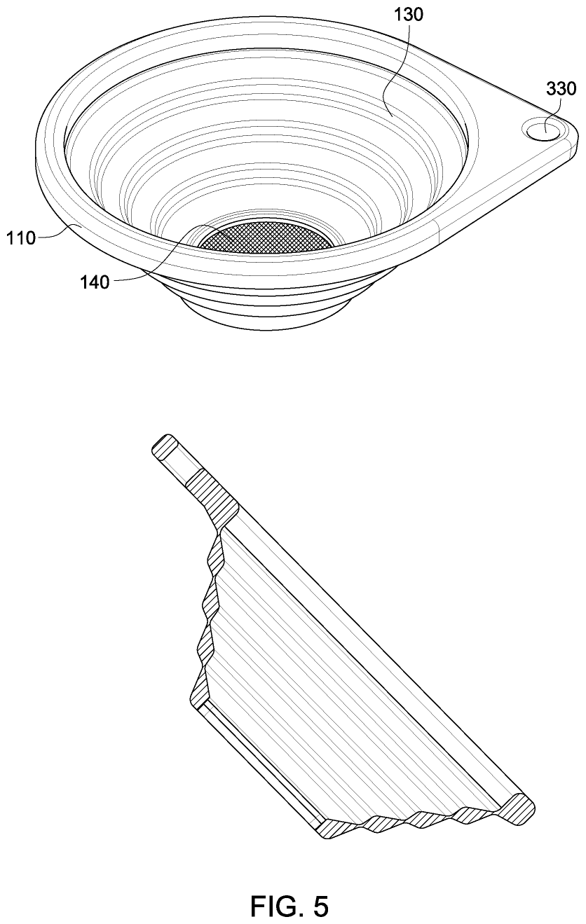

Referring to the drawings, wherein like reference numerals refer to like elements, FIGS. 1-5 illustrate embodiments of a fluid straining apparatus 100 configured to capture particulate matter from a fluid while permitting the fluid to pass therethrough.

In some embodiments, the fluid straining apparatus 100 includes an outermost ring portion 110 defining a top frame. The outermost ring portion 110 may be dimensioned to provide structural support and to facilitate positioning of the apparatus relative to a fluid stream. The outermost ring portion 110 may have a thickness 112 sufficient to provide rigidity and user handling comfort, and in some embodiments may include a tab 320 extending outwardly from an outer perimeter thereof.

The apparatus 100 further includes a plurality of ring portions telescopically coupled to one another and to the outermost ring portion 110 via sleeve portions 130. Each sleeve portion 130 extends between adjacent ring portions and permits relative sliding movement between the ring portions. The sleeve portions 130 may have a thickness that is less than the thickness of the ring portions to facilitate telescopic expansion and contraction. In some embodiments, the sleeve portions 130 may have a minimum thickness of approximately 0.5 mm at a thinnest portion to balance flexibility and durability.

An innermost ring portion 120 is disposed at a distal end of the telescoping assembly and is coupled to a mesh filter 140. The innermost ring portion 120 may have a thickness 122 sufficient to support and retain the mesh filter 140 during use.

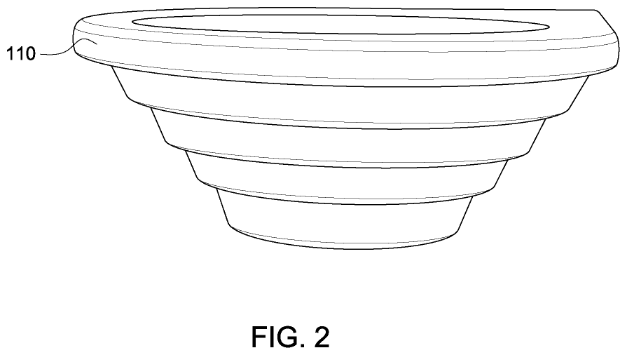

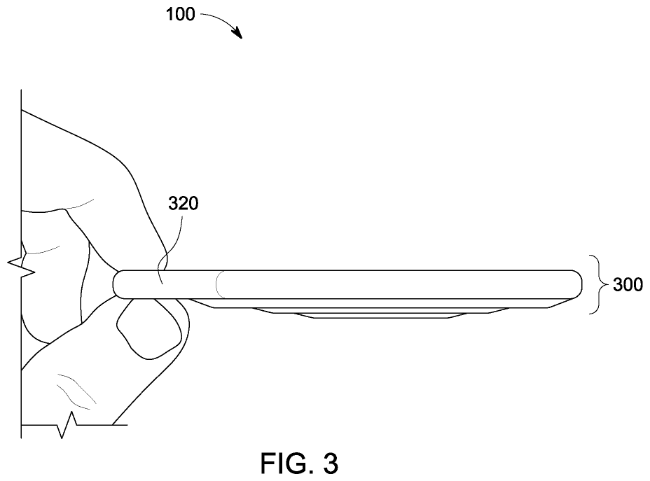

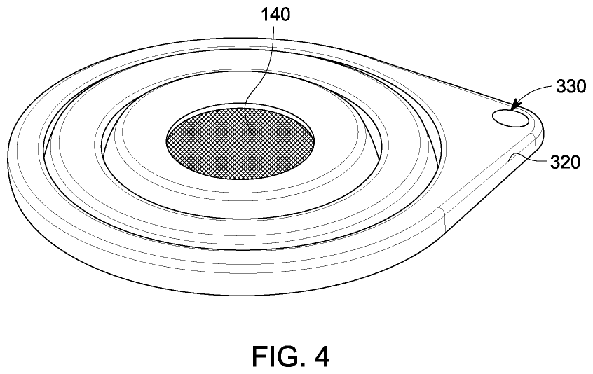

The plurality of ring portions are movable between a collapsed configuration (FIGS. 3-4) and an extended configuration (FIGS. 1-2). In the collapsed configuration, the ring portions are nested within one another to minimize overall size. In the extended configuration, the ring portions are sequentially extended to increase the effective filtration area.

In some embodiments, each ring portion defines an outer diameter and an inner diameter. Example dimensions for a five-ring configuration include approximate outer diameter/inner diameter pairs of: about 101.6 mm/89.2 mm; about 89.2 mm/76.4 mm; about 76.4 mm/63.7 mm; about 63.7 mm/50.9 mm; and about 50.9 mm/34.1 mm. These dimensions are illustrative and may vary depending on application. Each ring portion may have a maximum thickness between about 4 mm and about 7 mm, providing sufficient structural integrity while maintaining compactness.

The telescoping motion may be achieved by sequentially sliding adjacent ring portions relative to one another, as recited in the method claims.

In some embodiments, one or more of the ring portions and/or sleeve portions are formed from an elastomeric material such as silicone. The silicone may have a durometer hardness in the range of about Shore A 60 to about Shore A 70, providing a balance between flexibility, durability, and structural retention.

Alternative materials may include thermoplastic elastomers (TPE), rubber, or other polymeric materials exhibiting similar mechanical properties.

The mesh filter 140 is configured to permit passage of fluid while retaining particulate matter. In some embodiments, the mesh filter 140 is a 40-mesh filter, although other mesh sizes may be used depending on the desired filtration level.

In some embodiments, the mesh filter 140 is permanently secured to the innermost ring portion 120. For example, the innermost ring portion 120 may define a circumferential groove into which the mesh filter 140 is seated. The mesh filter 140 may be secured within the groove using a silicone adhesive or similar bonding material.

In alternative embodiments, the mesh filter 140 may be removably coupled to the innermost ring portion 120. For example, the mesh filter 140 may be retained by an interference fit, snap-fit engagement, threaded coupling, or removable retaining ring, thereby allowing replacement of the mesh filter.

In some embodiments, the apparatus 100, when in the collapsed configuration, has collapsed total height 300 of less than about 12 mm. This compact configuration enables convenient storage, portability, and attachment to personal items such as keychains or bags.

In some embodiments, the plurality of ring portions are configured to retain both the collapsed and extended configurations without requiring continuous external force. This self-retention may be achieved through frictional engagement between adjacent ring portions, interference fit between sleeve portions and ring portions, material elasticity (e.g., silicone restoring forces), or dimensional tolerances that resist unintended movement. In some embodiments, surface textures or slight radial interference may increase frictional retention.

In some embodiments, the fluid straining apparatus is configured to retain both the collapsed configuration and the extended configuration without requiring continuous external force. Such self-retention may be achieved by a combination of material selection and geometric configuration of the ring portions and sleeve portions. For example, one or more of the ring portions and sleeve portions may be formed from silicone having a durometer hardness of from about Shore A 60 to about Shore A 70. This hardness range may provide sufficient resilience to permit repeated telescopic movement while also providing enough stiffness and frictional resistance to inhibit unintended collapse from the extended configuration and unintended expansion from the collapsed configuration. In some embodiments, the sleeve portions have a reduced thickness relative to the adjacent ring portions, and may have a thickness of about 0.5 mm at a thinnest point, such that the sleeve portions flex during telescopic movement while the ring portions maintain structural support. The selected Shore hardness, together with the reduced sleeve thickness and the relative dimensions of adjacent ring portions and sleeve portions, may create a frictional and elastic interaction that causes the apparatus to remain in a selected configuration until an intentional force is applied by the user to extend or collapse the apparatus. Thus, in certain embodiments, the apparatus is self-retaining in both the collapsed and extended configurations by virtue of the material elasticity, dimensional tolerances, and resistance to deformation provided by the selected silicone hardness and sleeve geometry.

In some embodiments, the outermost ring portion 110 includes a tab 320 extending outwardly from its perimeter. The tab 320 may define an aperture configured to receive a hook, ring, or other attachment mechanism. This feature enables the apparatus to be coupled to a support structure during use or to a key ring, hook, or similar object during storage.

In use, the fluid straining apparatus 100 may be provided in a collapsed configuration. The user may extend the plurality of ring portions by sequentially sliding adjacent ring portions relative to one another until the apparatus reaches an extended configuration.

The outermost ring portion 110 may then be positioned relative to a fluid stream, such as a urine stream. The user may support the apparatus by holding the outermost ring portion 110 or by attaching the apparatus to a support structure via the tab 320.

Fluid is directed through the mesh filter 140 such that the fluid passes through the mesh while particulate matter is retained on the mesh surface. After use, the apparatus may be collapsed by reversing the telescoping motion, returning the ring portions to the nested configuration.

During storage, the apparatus may be coupled to a key ring or hook via the tab aperture 330, allowing convenient transport.

While specific embodiments have been described, variations may include different numbers of ring portions, alternative materials, different mesh sizes, and alternative attachment or retention mechanisms. The apparatus may be adapted for use with various fluids beyond urine, including other bodily fluids or liquids requiring particulate filtration.

Although certain embodiments are described herein in the context of straining urine or other bodily fluids, it will be understood that the disclosed fluid straining apparatus is not limited to such applications. The apparatus may be used for a wide variety of filtration and straining applications involving liquids and suspensions. For example, the apparatus may be used in food and beverage preparation, including but not limited to pour-over coffee filtration, loose leaf tea straining, cocktail or wine straining, flour sifting, and matcha preparation. In other embodiments, the apparatus may be used in outdoor, camping, or recreational settings, including filtration of liquids during travel or use in compact environments such as recreational vehicles. The apparatus may also function as a general-purpose portable sieve for filtering small particulate matter from fluids. Accordingly, the disclosed structure is not limited to any particular field of use, and references to specific applications are provided for illustrative purposes only.

In some embodiments, the ring portions and sleeve portions may be formed from silicone as described above. However, the disclosed apparatus is not limited to silicone materials. In alternative embodiments, one or more components of the apparatus may be formed from thermoplastic elastomers (TPE), thermoplastic polyurethane (TPU), rubber, or other flexible or semi-flexible polymeric materials capable of providing sufficient elasticity, durability, and structural retention to permit repeated telescopic movement. Material selection may be based on desired flexibility, chemical resistance, manufacturability, or cost considerations, and combinations of materials may also be employed.

The mesh filter may be selected from a range of mesh sizes depending on the intended filtration application. While certain embodiments utilize a mesh filter having a mesh size of approximately 40 mesh, other embodiments may include mesh sizes ranging from about 10 mesh to about 400 mesh or more. Lower mesh values may be used for coarse filtration of larger particulates, while higher mesh values may be used for finer filtration of smaller particles. The use of a separate mesh filter coupled to the innermost ring portion, rather than apertures formed directly in the ring material, enables selection of a wide range of mesh sizes and materials to achieve desired filtration characteristics.

In some embodiments, the sleeve portions extend between adjacent ring portions at an inward angle relative to a longitudinal axis of the apparatus, such that the ring portions collectively define a tapered or funnel-shaped geometry when in the extended configuration. This tapered configuration may facilitate directing fluid flow toward the mesh filter and improving filtration efficiency. The angle and degree of taper may vary depending on the relative dimensions of the ring portions and sleeve portions, and may be uniform or non-uniform across the plurality of ring portions.

What is claimed is:

1. A fluid straining apparatus configured to capture particulate matter from fluid, comprising:
an outermost ring portion defining a top frame;
a plurality of ring portions telescopically coupled to the outermost ring portion and to one another by sleeve portions, each sleeve portion extending between adjacent ring portions, each of the plurality of ring portions being movable between a collapsed configuration and an extended configuration; and
an innermost ring portion coupled to a mesh filter, the mesh filter being configured to permit passage of fluid therethrough while retaining particulate matter.

2. The fluid straining apparatus of claim 1, wherein at least a portion of the plurality of ring portions is formed from silicone.

3. The fluid straining apparatus of claim 2, wherein the silicone has a durometer hardness of from about Shore A 60 to about Shore A 70.

4. The fluid straining apparatus of claim 2, wherein adjacent ring portions are coupled by sleeve portions, each sleeve portion extending between adjacent ring portions and having a thickness less than a thickness of the ring portions to facilitate telescopic movement.

5. The fluid straining apparatus of claim 1, wherein the mesh filter is a 40 mesh filter.

6. The fluid straining apparatus of claim 1, wherein the outermost ring portion includes a tab extending outwardly from an outer perimeter of the outermost ring portion.

7. The fluid straining apparatus of claim 6, wherein the tab defines an aperture configured to receive a hook or a ring to facilitate coupling of the fluid straining apparatus thereto.

8. The fluid straining apparatus of claim 1, wherein, in a collapsed configuration, the plurality of ring portions each define an outer diameter and an inner diameter, and wherein the plurality of ring portions, from outermost to innermost, have approximate outer diameter and inner diameter pairs of: about 101.6 mm and 89.2 mm; about 89.2 mm and 76.4 mm; about 76.4 mm and 63.7 mm; about 63.7 mm and 50.9 mm; and about 50.9 mm and 34.1 mm.

9. The fluid straining apparatus of claim 4, wherein each sleeve portion has a thickness of about 0.5 mm at a thinnest point thereof.

10. The fluid straining apparatus of claim 1, wherein the plurality of ring portions each have a maximum thickness of between about 4 mm and about 7 mm.

11. The fluid straining apparatus of claim 1, wherein the mesh filter is seated within a groove defined by the innermost ring portion and is secured within the groove using a silicone adhesive.

12. The fluid straining apparatus of claim 1, wherein, in the collapsed configuration, the apparatus has a height of less than about 12 mm.

13. The fluid straining apparatus of claim 1, wherein the plurality of ring portions are configured to retain the collapsed configuration and the extended configuration without application of continuous external force.

14. The fluid straining apparatus of claim 1, wherein the mesh filter is removably coupled to the innermost ring portion to permit replacement of the mesh filter.

15. A method of straining fluid using a fluid straining apparatus, the apparatus including an outermost ring portion defining a top frame, a plurality of ring portions telescopically coupled to one another, and an innermost ring portion coupled to a mesh filter, the method comprising: providing the fluid straining apparatus in a collapsed configuration; extending the plurality of ring portions from the collapsed configuration to an extended configuration; positioning the outermost ring portion relative to a fluid stream; and directing fluid through the mesh filter such that fluid passes through the mesh filter and particulate matter is retained by the mesh filter.

16. The method of claim 15, wherein extending the plurality of ring portions comprises sequentially sliding adjacent ring portions relative to one another.

17. The method of claim 15, further comprising supporting the fluid straining apparatus by the outermost ring portion during urination.

18. The method of claim 15, further comprising collapsing the plurality of ring portions from the extended configuration to the collapsed configuration after use.

19. The method of claim 15, further comprising coupling the fluid straining apparatus to a support structure via a tab extending from the outermost ring portion prior to directing fluid through the mesh filter.

20. The method of claim 15, further comprising, during storage, coupling the fluid straining apparatus to a key ring or a hook via an aperture defined in a tab extending from the outermost ring portion.
